(19) United States
(12) Patent Application Publication
(10) Pub. No.: US 2012/0141467 A1
(43) Pub. Date: Jun. 7, 2012

(54) Ascorbic acid to treat chronic obstructive lung diseases and non-Hodgkin's lymphoma

(76) Inventor: Daniel J. Schneider, Grapevine, TX (US)

(21) Appl. No.: 12/928,095

(22) Filed: Dec. 3, 2010

(51) Int. Cl. A61K 31/375; A61K 31/355; A61K 31/616; A61K 31/405; A61K 31/541; A61P 11/00; A61K 31/65; A61K 38/12; A61K 31/43; A61K 39/395; A61K 31/473; A61P 35/00; A61K 31/56; A61K 31/542

(52) U.S. Cl. 424/133.1; 514/474; 514/171; 514/458; 514/161; 514/420; 514/222.2; 514/226.5; 514/154; 514/2.9; 514/192; 424/141.1; 514/290

(57) ABSTRACT

Provided herein is a method for treating a tracheo-bronchial-alveolar tract disease in a subject in need thereof, the method comprising the step of administering to a subject in need of such treatment a pharmaceutical composition comprising a therapeutically effective amount of ascorbate or a derivative thereof. Also provided is a method for treating non-Hodgkin's lymphoma in a subject in need thereof, the method comprising the step of administering to the subject in need of such treatment a pharmaceutical composition comprising a therapeutically effective amount of ascorbate or a derivative thereof.

### BACKGROUND OF THE INVENTION

1. Field of Invention

The present invention relates generally to the fields of pulmonary biochemistry and physiology as well as cancer biology. More specifically, the present invention relates to the use of ascorbic acid to treat chronic inflammatory/obstructive lung diseases and non-Hodgkin's lymphoma.

2. Description of the Related Art

Chronic lung diseases such as chronic obstructive pulmonary disease (COPD), cystic fibrosis (CF) and idiopathic pulmonary fibrosis (IPF) are a major global problem. COPD, in particular, is predicted to feature among the five most common causes of morbidity and mortality in the human population by 2020. COPD is characterized by one or more of the following activities; infiltration of leukocytes into the lungs, interstitial lung inflammation, increased pro-inflammatory cytokine production, thickening of lung alveolar setae, hemorrhagic pneumonitis, distortion of normal lung architecture and fibrosis brought about by increased collagen deposition coupled with altered protease activity. Moreover, disease progression results in significant loss of lung tissue and scarring ultimately leading to the substantial morbidity and mortality associated with these chronic lung diseases.

Presently, treatment of chronic lung diseases is limited to symptom alleviation and/or prevention (e.g., bronchodilators such as β2-agonists and theophylline, and/or anti-inflammatory corticosteroids such as prednisone, are used to improve breathing capacity), and accordingly, the development of drugs or treatments to arrest, or preferably, reverse disease progression are greatly awaited.

Malignant tumors (cancers) are the second leading cause of death in the United States, after heart disease. Cancer is characterized by the increase in the number of abnormal, or neoplastic, cells derived from a normal tissue which proliferate to form a tumor mass, the invasion of adjacent tissues by these neoplastic tumor cells, and the generation of malignant cells which eventually spread via the blood or lymphatic system to regional lymph nodes and to distant sites via a process called metastasis. In a cancerous state, a cell proliferates under conditions in which normal cells would not grow.

Cancers which involve cells generated during hematopoiesis, a process by which cellular elements of blood, such as lymphocytes, leukocytes, platelets, erythrocytes and natural killer cells are generated are referred to as hematopoietic cancers. Lymphocytes which can be found in blood and lymphatic tissue and are critical for immune response are categorized into two main classes of lymphocytes: B lymphocytes (B cells) and T lymphocytes (T cells), which mediate humoral and cell mediated immunity, respectively.

B cells mature within the bone marrow and leave the marrow expressing an antigen-binding antibody on their cell surface. When a naive B cell first encounters the antigen for which its membrane-bound antibody is specific, the cell begins to divide rapidly and its progeny differentiate into memory B cells and effector cells called “plasma cells”. Memory B cells have a longer life span and continue to express membrane-bound antibody with the same specificity as the original parent cell. Plasma cells do not produce membrane-bound antibody but instead produce the antibody in a form that can be secreted. Secreted antibodies are the major effector molecule of humoral immunity.

T cells mature within the thymus which provides an environment for the proliferation and differentiation of immature T cells. During T cell maturation, the T cells undergo the gene rearrangements that produce the T-cell receptor and the positive and negative selection which helps determine the cell-surface phenotype of the mature T cell. Characteristic cell surface markers of mature T cells are the CD3:T-cell receptor complex and one of the coreceptors, CD4 or CD8.

In attempts to discover effective cellular targets for cancer therapy, researchers have sought to identify transmembrane or otherwise membrane-associated polypeptides that are specifically expressed on the surface of one or more particular type(s) of cancer cell as compared to on one or more normal non-cancerous cell(s). Often, such membrane-associated polypeptides are more abundantly expressed on the surface of the cancer cells as compared to on the surface of the non-cancerous cells. The identification of such tumor-associated cell surface antigen polypeptides has given rise to the ability to specifically target cancer cells for destruction via antibody-based therapies. In this regard, it is noted that antibody-based therapy has proved very effective in the treatment of certain cancers. For example, HERCEPTIN® and RITUXAN® (both from Genentech Inc., South San Francisco, Calif.) are antibodies that have been used successfully to treat breast cancer and non-Hodgkin's lymphoma, respectively. More specifically, HERCEPTIN® is a recombinant DNA-derived humanized monoclonal antibody that selectively binds to the extracellular domain of the human epidermal growth factor receptor 2 (HER2) proto-oncogene. HER2 protein overexpression is observed in 25-30% of primary breast cancers. RITUXAN® is a genetically engineered chimeric murine/human monoclonal antibody directed against the CD20 antigen found on the surface of normal and malignant B lymphocytes. Both these antibodies are recombinantly produced in CHO cells.

In other attempts to discover effective cellular targets for cancer therapy, researchers have sought to identify (1) non-membrane-associated polypeptides that are specifically produced by one or more particular type(s) of cancer cell(s) as compared to by one or more particular type(s) of non-cancerous normal cell(s), (2) polypeptides that are produced by cancer cells at an expression level that is significantly higher than that of one or more normal non-cancerous cell(s), or (3) polypeptides whose expression is specifically limited to only a single (or very limited number of different) tissue type(s) in both the cancerous and non-cancerous state (e.g., normal prostate and prostate tumor tissue). Such polypeptides may remain intracellularly located or may be secreted by the cancer cell. Moreover, such polypeptides may be expressed not by the cancer cell itself, but rather by cells which produce and/or secrete polypeptides having a potentiating or growth-enhancing effect on cancer cells. Such secreted polypeptides are often proteins that provide cancer cells with a growth and/or survival advantage over normal cells and include such things as, for example, angiogenic factors, cellular adhesion factors, growth factors, and the like. Identification of antagonists of such non-membrane associated polypeptides would be expected to serve as effective therapeutic agents for the treatment of such cancers. Furthermore, identification of the expression pattern of such polypeptides would be useful for the diagnosis of particular cancers in mammals.

Non-Hodgkin lymphoma is any of a large group of cancers of lymphocytes. Non-Hodgkin lymphomas are noted by enlarged lymph nodes, fever, and weight loss. Non-Hodgkin lymphoma are divided into aggressive and indolent types, and they can be formed from either B-cells or T-cells. B-cell non-Hodgkin lymphomas include Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. T-cell non-Hodgkin lymphomas include mycosis fungoides, anaplastic large cell lymphoma, and precursor T-lymphoblastic lymphoma. Lymphomas that occur after bone marrow or stem cell transplantation are usually B-cell non-Hodgkin lymphomas. In 2010, there were estimated to be approximately 65,000 new cases of non-Hodgkin lymphoma in the United States.

Despite the above-identified advances in mammalian cancer therapy, there is a great need for additional therapeutic agents capable of detecting the presence of tumor in a mammal and for effectively inhibiting neoplastic cell growth and/or survival, respectively. The present invention fulfills this longstanding need and desire in the art.

### SUMMARY OF THE INVENTION

The present invention is directed to a method for treating a tracheo-bronchial-alveolar tract disease in a subject in need thereof, the method comprising the step of administering to a subject in need of such treatment a pharmaceutical composition in the form of an aerosol inhalant comprising a therapeutically effective amount of ascorbate or a derivative thereof.

The present invention is further directed to a nasal or oral inhalant comprising an aqueous carrier having dissolved therein ascorbate or a derivative thereof.

The present invention is further directed to a method for treating non-Hodgkin's lymphoma in a subject in need thereof, the method comprising the step of administering to the subject in need of such treatment a pharmaceutical composition in the form of an aerosol inhalant comprising a therapeutically effective amount of ascorbate or a derivative thereof.

### BRIEF DESCRIPTION OF THE DRAWINGS

FIGS. 1A-1C show a respiratory remissions graph (FIG. 1A) and rehabilitation graph (FIGS. 1B-1C). Graphing the cross-over results of the respiratory morbidity signs plus prescribed medication data in Table 1 is presented.

FIG. 2 shows a lung mucosa effects graph.

### DETAILED DESCRIPTION OF THE INVENTION

In one embodiment of the present invention, there is provided a method for treating a tracheo-bronchial-alveolar tract disease in a subject in need thereof, the method comprising the step of administering to a subject in need of such treatment a pharmaceutical composition comprising a therapeutically effective amount of ascorbate or a derivative thereof. Representative tracheo-bronchial-alveolar tract diseases included but are not limited to chronic obstructive pulmonary disease, inflammatory lung diseases, bronchitis, including chronic bronchitis and emphysema. Given the information provided herein, a person having ordinary skill in this art could readily ascertain the appropriate dosages and/or formulations of ascorbate. Generally, the composition contains from about 1 g to about 20 g of ascorbate or a derivative thereof per 100 ml and more preferably from about 5 g to about 15 g of ascorbate or a derivative thereof per 100 ml. Representative forms of the formulation include but are not limited to the composition being contained in an aerosol or nebulized inhalant or a lozenge.

In another embodiment of the present invention, there is provided a nasal or oral inhalant comprising an aqueous carrier having dissolved therein ascorbate or a derivative thereof. Preferably, the aqueous carrier comprises from about 5 to about 15 wt. % of ascorbate. It is contemplated that the nasal or oral inhalant of the present invention could be administered concurrently with another drug, such as but not limited to corticosteroids, antihistamines, non-steroidal anti-inflammatory agents, anti-cholinergics, expectorants, antibiotics, histamine H₃ receptor antagonists, leukotriene D₄ antagonists, leukotriene inhibitors, P₂Y agonists, syk kinase analogues and vitamin E. Representative non-steroidal anti-inflammatory agent include acetyl salicylic acid, acetaminophen, indomethacin, diclofenac, piroxicam, tenoxicam, ibuprofen, naproxen, ketoprofen, nabumetone, ketorolac, azapropazone, mefenamic acid, tolfenamic acid, sulindac, diflunisal, tiaprofenic acid, podophyllotoxin derivatives, acemetacin, aceclofenac, droxicam, oxaprozin, floctafenine, phenylbutazone, proglumetacin, flurbiprofen, tolmetin, and fenbufen. Representative antibiotics include tetracycline, chlortetracycline, bacitracin, neomycin, polymyxin, gramicidin, oxytetracycline, chloramphenicol, flofenicol, gentamycin, erythoromycin, clarithromycin, azithromycin, tulathromycincefurpxo, ceftobitem, ceftiofur, defadroxil, amoxicillin, penicillin, amoxicillin combined with a beta-lactamase inhibitor, sulfonamides, sulfacetamide, sulfamethizole, sulfisoxazole, nitrofurazone, and sodium propionate.

In another embodiment of the present invention, there is provided an inhalable composition suitable for use in a nebulizer comprising a ascorbate wherein said ascorbate is packaged for simultaneous, sequential, or separate inhalation administration of at least one additional medicament comprising a solution or suspension containing at least one member of the group consisting corticosteroids, antihistamines, non-steroidal anti-inflammatory agents, anti-cholinergics, pharmaceutically acceptable zinc salts, expectorants, antibiotics, histamine H₃ receptor antagonists, leukotriene D₄ antagonists, leukotriene inhibitors, P₂Y agonists, syk kinase analogues, vitamin E and combinations of two or more thereof.

In another embodiment of the present invention, there is provided a method of treatment of an upper or lower respiratory, viral, inflammatory, or obstructive airway disease comprising administration of an effective amount of a composition provided herein. This administration could be, e.g., carried out using a nebulizer.

In another embodiment of the present invention, there is provided a method for treating non-Hodgkin's lymphoma in a subject in need thereof, the method comprising the step of administering to the subject in need of such treatment a pharmaceutical composition comprising a therapeutically effective amount of ascorbate or a derivative thereof. Preferably, this composition contains from about 5 g to about 15 g of ascorbate or a derivative thereof per 100 ml. It is specifically contemplated that this method may further comprise concurrently administering a compound selected from the group consisting of a aza-anthracenedione, rituximab, ibritumomab and tositumomab. Preferably, the composition is administered to respiratory mucosa or lymphatic tissue of said subject and the composition is in the form of an aerosol inhalant or a lozenge.

### CO-ILD-Ecoloqv Model:

The CO-ILD-Ecology Model factors Zoological::Host::Agent::Environment etiology into the multivariate pool of life-long lung inflammation and obstruction dysfunction coefficients in the context of 1) genotypic; 2) phenotypic; 3) pathologic; and 4) logistic variables, gaining insight from immunologic and molecular and biological knowledge that evidences ascorbate (ASC) reagent roles in lung parenchyma and mucociliary health/disease domain. The therapeutic goal is to assess access and concentration benefits of direct topical application of ASC to respiratory mucosa/lymphatic tissue in its role as an essential reagent continuously consumed in multiple molecular paths for lung health.

Specific to CO-ILD, the model prescribes upper and lower respiratory system mucosa topical application experiments to follow preliminary tests of ascorbate applied topically to mucosa of the nose and mouth. Safety tests results with significant mucosa benefits and no toxic mucosa effects using significant concentration of ascorbate, first in sterile water and then in a lozenge were predicted and observed. The model predicts an ascorbate formula for daily aerosolized inhalation, with divided dosing to be efficacious and safe in a range of plausible ascorbate per day in increments, each increment being delivered in a manageable yet physiological response defined duration of time. The dose volume is suggested at 3 to 7 ml using a conventional nebulizer with the aerosol being slowly inhaled (as a function of the nebulizer in use) followed by very rapid complete forced exhalation to expel as much trapped air as possible given that normal exhalation is passively accomplished by pulmonary elasticity, not muscular contraction. Within an appropriate clinical trial protocol, it is probable that any CO-ILD subject need have conventional combined treatments, i.e. antibiotic, anti-inflammation, expectorant, broncho-dilatation, and immune support medications, for bronchitis, obstruction, acute infections/exacerbations, and any asthma and/or pneumonia complications attenuated as indicated by disease progress. The ascorbate aerosol is named “Schneider's Inhalant”, or SI. The lozenge adopted the name “Schneider's Troche”, called ST.

### The Index Case:

The Index Case (IC) is a 77 year old Caucasian male with 50+ year history of chronic lung disease with unrelenting progression manifest by chronic bronchitis with acute bronchitis events; chronic productive cough, dyspnea, asthma, and repeated pneumonia events. His progression lung disease diagnoses include acute and chronic bronchitis and pneumonia (on occasion requiring hospitalization), chronic obstructive disease, asthma, and restrictive lung dysfunction. Co-morbidities commenced with childhood atopy and Meniere's disease. As an adult, co-morbidities include primary immune deficiency of unknown duration or variability, chronic gingivitis and stomatitis with frequent papilloma and herpes virus events, chronic fungal dermatitis, intermittent peptic ulcer, cholecystitis (excised 1994), prostate cancer (excised 1998), and non-Hodgkin's lymphoma since 1998.

The IC is a generally cognizant physician with extensive chest diseases clinical experience. As his own pulmonary conditioned worsened, a CO-ILD ecological model was formulated followed by organizing and execution of the clinical experiment herein reported. Based on hypotheses prescribed by the model, and experiments were designed and first upper and then lower respiratory mucosa applications of ascorbate were assessed, including the respiratory aerosol formulation Schneider's Inhalant (SI).

### CO-ILD Differential:

The Index case disease condition reflects both obstructive and restrictive lung dysfunctions. The index case had never been a steady smoker. During the early years of his chronic disease, frequent asthma attacks combined with chronic bronchitis and resulted in the index case's condition being frequently interpreted clinically as impending chronic obstructive disease because obstruction from inflammation was not substantially reduced by bronchodilation during spirometry.

During the 1980s, asthma attacks persisted, requiring frequent use of bronchodilators. The chronic bronchitis intensified with chronic production of purulent sputum, and pneumonitis was a frequent occurrence. Frequent use of antibiotics was required. Coughing became strident and mucous was difficult to clear. Chest discomfort became persistent with dyspnea being ever more incapacitating. Restrictive lung dysfunction became the dominant presumptive diagnosis.

“Normal Respiratory Flora” was a typical sputum culture description. Intense night sweats and low grade fever were chronic and gradually intensified during the late 1970s and 1980s. By the 1990s, prophylactic antibiotics were prescribed in an attempt to reduce pneumonia recurrence. Spirometry confirmed restrictive lung dysfunction.

### Index Case Respiratory Disease History:

As a young person the IC had severe “hay fever” with rhinitis, bronchitis, and ear infections diagnosed by skin testing as due to multiple allergies (pollens and danders). At about age 25, an acute, severe bronchitis event progressed to lobar pneumonia, requiring hospitalization for more than a week. Subsequent to the pneumonia event, the IC had residual chronic bronchitis with mild asthma, which progressed to severe bronchitis events with occasional severe asthma, protracted strident coughing, and superimposed pneumonitis/pneumonia events.

By the mid-1990s, the IC had intractable broncho-pulmonary obstruction, inflammation and lung infections. His chronic, persistent cough produced thick discolored sputum and tenacious mucous plugs, and he experienced many repeated pulmonary infections, dyspnea, tachypnea, nightly drenching sweats, constant rales (audible unaided), air trapping with rhonchi and audible sqeeks, frequent bouts of pneumonia requiring many repeated courses of antibiotic therapy. His vocal chords became congested and his voice “gravely”. His condition continually worsened and in 1997, his allergist interpreted restrictive lung disease by flow volume loop spirometry. By this time, the IC was on prophylactic antibiotic maintenance, required frequent albuterol inhalation, intermittent theophylline, and his FVC became progressively reduced to 3.25-liters. Provoked and frustrated by pneumonia events between 1991-8 with no relief in sight by use of conventional medical treatment, the IC created the ascorbate CO-ILD Ecology Model † whereupon he formulated and initiated his original and unique ascorbate CO-ILD treatment experiment.

### Index Case ASC CO-ILD Treatment Study Summary:

The IC is now in year eighteen of the CO-ILD Ecology Model and ASC treatment experiment. A summary of disease and treatment experiment progress to present is as follows:

1950˜1991—Progressive worsening of lung disease—chronic productive cough, obstruction, frequent infections.
1991˜1998—Regular work schedule impossible; ASC oral and upper respiratory mucosa treatment assessment, including mucosa safety and toxicity assessment—coincidental diagnose of generalized grade IV NHL in 1998.
1998˜2002.11—confirmed restrictive lung dysfunction; performed 2 empirical ASC SI inhalation experiments and 4 cross-over ascorbate SI experiments; —incidental observed NHL suppression during cross-over ascorbate SI experiments.
2002.11˜2005.6—confirmed restrictive lung dysfunction in remission; 2.5 years daily ascorbate SI maintenance, remission of both lung and NHL symptoms and signs.
2005.6˜2006.8—1 year with no pulmonary symptoms or signs while abstaining ASC SI; 2006.7 pet scan confirms NHL reactivation, NHL nodes reappear at rt clavicle and rt sub mandible; 2006.8 excise sub mandible node—pathology Dx small/large cell transformation; fresh node delivered for culture; culture fails.
2006.8˜2007.6—1 year reinstitute ASC SI; clavicle nodes disappear; CT 28mar2007 all nodes normal size.
2007.6˜2007.7—train 1 year to successfully run San Francisco Marathon; maintain ASC SI.
2007.7˜2008.6—continue exercise; full time work, averaging work 12-16 h/d; Surveilance PET scan reveals residual interstitial lung disease, no abnormal lymph tissue; therefore start cross-over—abstain ASC SI.
2008.6˜2010.10—intermittent light râies, occasional clear sputum; no palpable abnormal lymphatic tissue; maintain cross-over—abstain ASC SI.
2010.10˜present—maintenance CT on 8 Oct. 2010 reveals enlarged mediastinal nodes; no palpable abnormal lymphatic tissue; cross-over nebulized ascorbate SI reinstated; repeat CT comparison on 5 Nov. 2010 reveals enlarged nodes reduced in size by as much as 60%. Cross-over ascorbate SI to be maintained.

### Index Case ASC CO-ILD Cross-over Studies:

To date, two cross-over experiments have been put in play:

1) 1999˜2003—establish efficacy and significance of ascorbate SI to treat chronic obstructive and interstitial lung disease.
2) 2003˜present—explore and document efficacy and significance of role of ascorbate SI to treat NHL—have experienced two successful cross-over tests and currently observing third cross-over with satisfactory clinical progress.

### ASC Aerosol Experiment:

After assessing the safety and toxicity of ASC from 1991 to 1997, the IC formalized an informed consent to test feasibility experiments to use ascorbate aerosol to treat his CO-ILD. The experiments were designed to test the plausibility of using ascorbate aerosol as a topically applied complementary mucosa “nutraceutical” to alter the course of acute exacerbations of chronic bronchitis. Initiation of the test sought first to assure that the designed aerosol would be safely tolerated by the IC's respiratory mucosa. In 1998 the experiments explored topical ascorbate mucosa application to the upper respiratory mucosa, at which locations knowledge of natural tolerance for topical ascorbate is well established. Several concentrations of ascorbate in defined by mucosa response testing were formulated in sterile water and were tested in direct application to the IC's upper respiratory mucosa, applied first as an oral spray and then as a nasal spray. The applications were tasteless and odorless, giving the IC no discomfort. Three ml spray applications to the upper respiratory mucosa were continued three times daily for six months with no adverse effects. The IC did note, however, that chronic gingivitis was relieved by these applications and any early upper respiratory infection (URI) prodrome occurrences would be promptly interrupted by the topical spray applications thereby stopping URI occurrences that historically were precursors to acute bronchitis exacerbations.

During a severe acute bronchitis exacerbation in May, 1998, for the first time (“Start SI Rx” on graphs below) the IC inhaled 3 ml of a nebulized solution of ascorbate formulated in sterile water in compliance with upper respiratory safety tests noted previously. The SI was aerosolized by means of a conventional nebulizer, the SI being slowly and steadily inhaled followed by very rapid, forced, exhalation completed as much as possible, accompanied by pronounced squeaks, sputum expectoration, and rhonchi. The 3 ml SI inhalation was repeated three times daily. The IC quickly and dramatically improved; by the 10^{th} day the acute bronchitis episode had resolved and the IC's chronic bronchitis status was re-established. Thus the aerosol nebulizations were discontinued on day 10. The formulation became termed “Schneider's Inhalant” (SI). In May 1999 immunologist performed spirometry confirmed the IC current status of chronic restrictive lung dysfunction persisted while symptoms were improved. Another severe acute bronchitis exacerbation occurred in November, 1999, and another in April, 2000; and the experiment was repeated with the same beneficial result. During the next three years, a series of four additional cross-over experiments were performed in which the IC would immediately restart SI aerosol administration at the time of each acute bronchitis exacerbation followed by prompt remission and no adverse side effects. No deliberate changes were made in physician prescribed lung obstructive disease medication protocols, use of which during chronic bronchitis periods was essentially driven by PRN instructions; of note, all prescribed bronchodilators, expectorants, mucolytics, and antibiotics were not required to maintain normal lung function. On 10 Dec. 2002, the immunologist repeated the spirometry which showed that the restrictive lung dysfunction was in complete remission.

### ASC Aerosol Experiment Results:

Cross-over controlled experiment is defined as the experiment subject and the experiment control being the same person; in this case being the IC. Thus, in this experiment the IC served as subject by measured observation of severity during acute experience of acute bronchitis exacerbation events in which no SI was applied (second column in Table I), and followed by severity comparison during acute exacerbations in which SI was applied (third column in Table I). In the second comparison, the acute events were allowed to become temporally established based on morbidity signs and symptoms. The established acute event was then followed by parametric administration of the SI treatment with observation and recording of any change in morbid signs and symptom, measuring severity and end-point results to quantify any temporally associated reversal and recovery from the acute event with return to “stable” chronic bronchitis status. The IC then serving as control during “stable” chronic periods with non-treatment (no SI aerosol in the absence of acute bronchial infection), such parametric non-treatment being sustaining until onset of a new acute bronchitis exacerbation with dyspnea, cough, and lower respiratory infection productive of greenish discolored mucous. Then, at the onset of the recurring acute bronchial infection, the IC would revert to being Subject and would again follow the SI nebulization protocol.

After four such uncomplicated cross-over controlled experiments, the IC's FVC had improved from 3.73 liters restrictive lung volume to normal 4.23 liters lung volume, and his pulmonary pathology symptoms and signs had returned to “normal”, with mild chronic bronchitis events such as intermittent persistent rales, chronic cough, and daily production of clear mucous plugs, and with no signs of obstruction or asthma. Neither maintenance antibiotics, nor any other medications for CO-ILD, have been required by the IC since 1999, except on one acute bronchitis occasion in 2001 when a single course of antibiotics was required when fever occurred as a complication of an acute bronchitis event.

All use of ASC SI since 1999 has been associated with management of the IC's co-morbid NHL. The use of ASC SI for the IC's NHL may mask any further exacerbation of CO-ILD.

### Analysis of IC Data

Following the fourth cross-over controlled experiment in 2002 (seventh ASC SI experiment), the IC clearly benefited the reversal of his historic CO-ILD with statistically significant improvement. All symptom and sign end-points had significant levels <0.05 (see Table I) of improvement with no adverse side effects being detected. These significance levels indicate a very low probability that the differences observed in these experiments were due to chance: frequency and severity of acute bronchitis events became controlled; asthma has stopped; no new pneumonia events occurred; measured respiratory function was rehabilitated to normal, and no sign of the progressive obstructive or inflammatory lung disease remained—after 40 years of unrelenting progression in severity. Complete elimination for need of antibiotic, anti-inflammatory, bronchodilatory, and expectorant therapy for the treatment of CO-ILD was realized.

TABLE I
Feasibility Study Result Significance: CO-ILD Index Case SI Treatment

| | Severity With | Severity With SI | | |
|---|---|---|---|---|
| | No SI Rx, avg | Rx ~4 experiments | Standard | Significance |
| | 8 events/yr | repeated in series | Morbidity | Level set at |
| Symptom/Sign/Treatment | during 1994-98 | during 1999-02 | Ratio | 0.05; P(T <= t) |
| Respiratory Morbidity Signs | | | | |
| Exp wheezes/squeaks (10~0)* | 7.0 | 3.0 | 0.60 | 0.0372 |
| Vital capacity; liters | 3.667 | 4.250 | 0.84 | 0.0056 |
| Night sweats; days/event | 54 | 0 | 0.10 | 0.0002 |
| Sick leave; days/event | 21 | 1 | 0.14 | <0.0001 |
| Mucosa Morbid Signs | | | | |
| Purulent sputum, AM, ml | 14.0 | 2.2 | 0.30 | 0.0008 |
| Bronchial casts (10~0)* | 9.0 | 1.75 | 0.27 | 0.0106 |
| Mucous fluidity (10~0)** | 1.0 | 9.5 | 0.13 | 0.0012 |
| Râles and rhonchi (10~0)* | 9.33 | 0.0 | 0.11 | 0.0001 |
| Medication Prescriptions | | | | |
| Antibiotics, days Rx/event | 20 (avg) | 3 (avg) | 0.25 | 0.0013 |
| Albuterol, 3 ml aerosols/day | 3 | 0 | 0.06 | <0.0001 |
| SI, 3 ml aerosol, days/event | 0 | 16.75 | 0.10 | <0.0001 |

Severity:
( )*10 = worst, 0 = best
( )**10 = best, 0 = worst.
Other data values as specified

Table 1 summarizes 11 of the 20 SI aerosol treatment end-point measures as compared to conventional treatment for chronic obstructive bronchitis events observations as they occurred in the IC between 1994 and 2002. Lozenge and upper respiratory spray safety assessed since 1994 with no occurrence of adverse side effects.

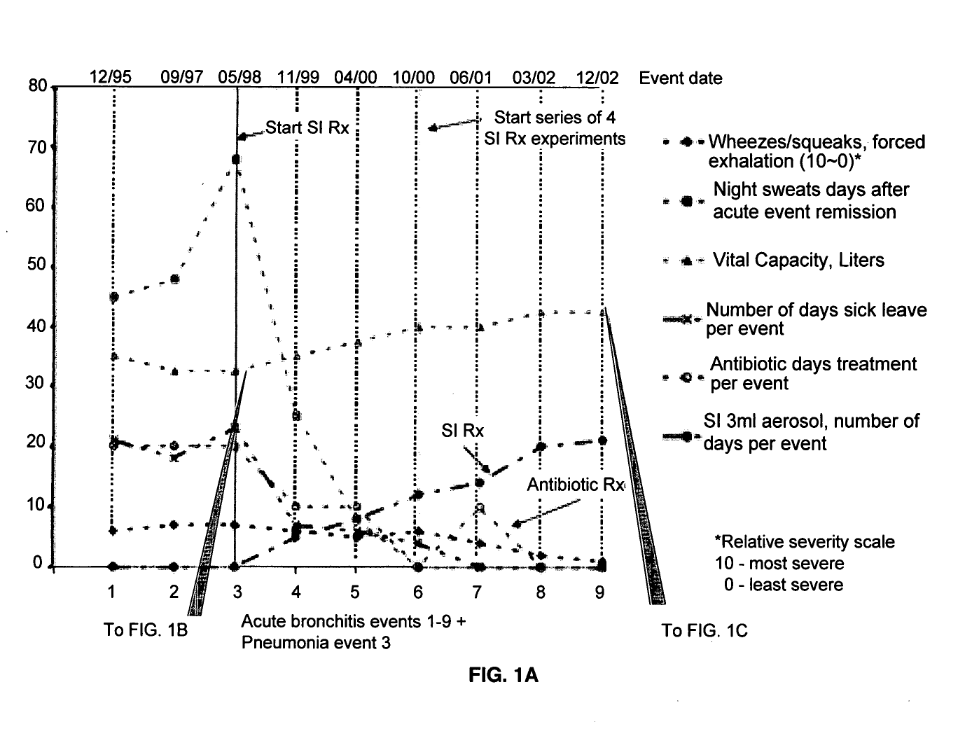

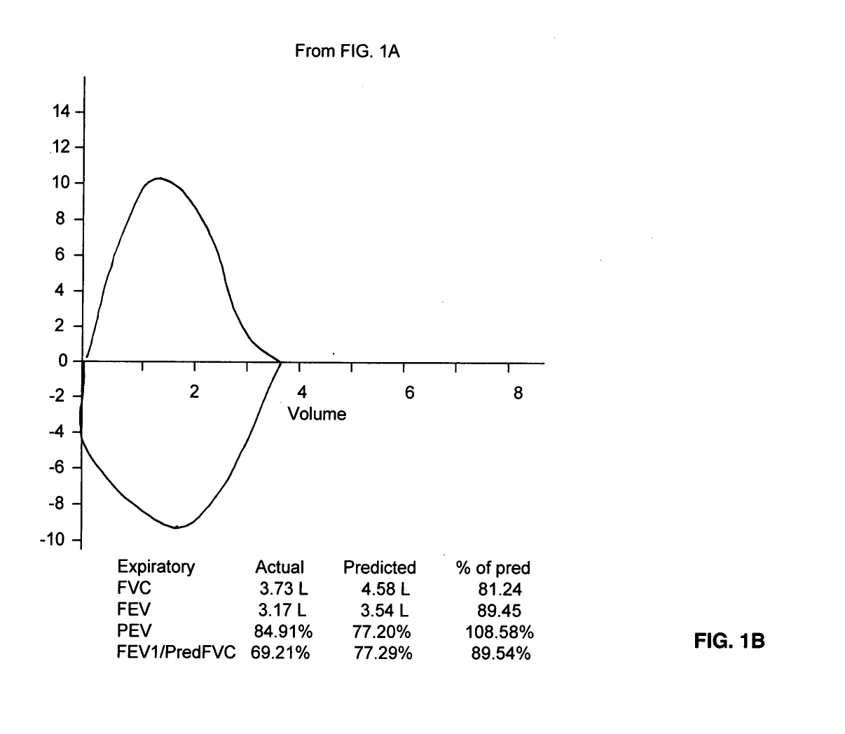

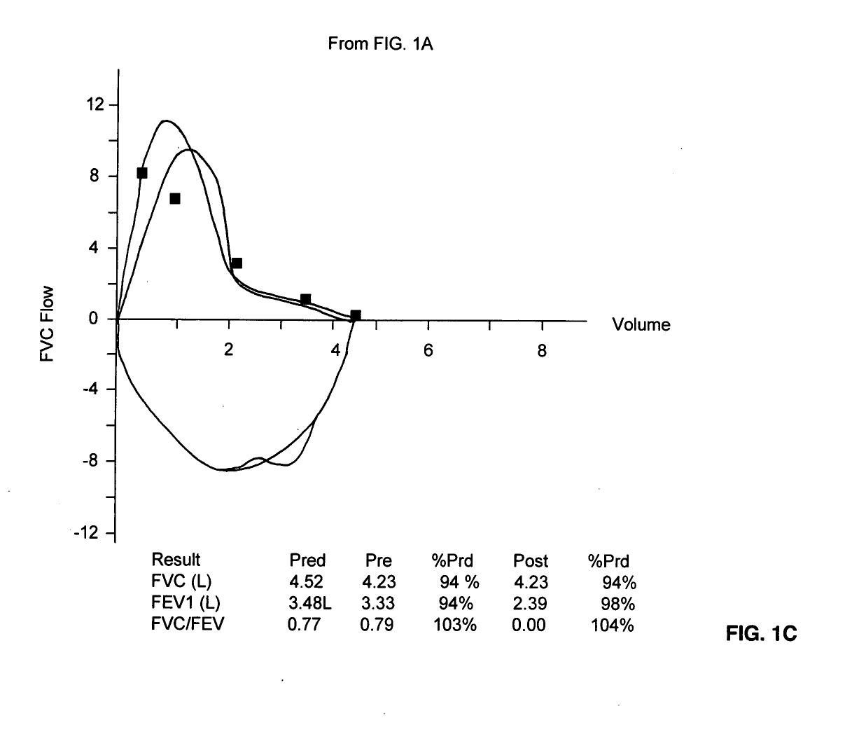

FIGS. 1A-1C show a respiratory remissions graph (FIG. 1A) and rehabilitation graph (FIGS. 1B-1C). Graphing the cross-over results of the respiratory morbidity signs plus prescribed medication data in Table 1 is presented. The IC was maintained on prophylactic antibiotics as well as conventional CO-ILD medications prior to event #3 in March, 1998, which was the last of numerous previous pneumonia recurrences and was the last pulmonary disease event in which convention CO-ILD remedies (i.e. Albuterol, expectorants) were required. The “Start SI Rx” line indicates when the decision was made to initiate SI experiments to treat any recurrence of acute pulmonary infections. Thus, observations and treatments experienced in events #4 and #5, occurring during 1999 and 2000, reflect the results during empirical SI treatment experiments. Events #6, #7, #8, and #9 occurred during 2000 to 2002, and graph the results of four simple reversal (cross over) experiments in which SI treatment was started at the onset of acute bronchitis symptoms and then continued for 3 weeks. After each experiment, the SI was stopped. Symptoms of chronic bronchitis would continue, and on the occurrence of the next acute bronchitis reoccurrence, the SI treatment protocol was repeated with rapid clearance of acute bronchitis being observed. Since June, 2001, following event #7, SI aerosol is started immediately at onset of acute bronchitis symptoms and no further antibiotics have been required. Following event #9 in November, 2002, the IC has maintained daily SI aerosol inhalations, and has had no further acute bronchitis events and has no chest disease experiences or conscious perception of chronic broncitis.

The use of SI correlated directly with the significant, progressive increase in lung forced tidal volume (vital capacity); and the increase in forced tidal volume correlates inversely with both the progressive decrease in persistent expiratory wheezes/squeaks and bronchiolar cast production, and likely represents a decrease in air trapping associated with reduced bronchiolar remodeling obstruction. These are impressive reversals in CO-ILD disease progression.

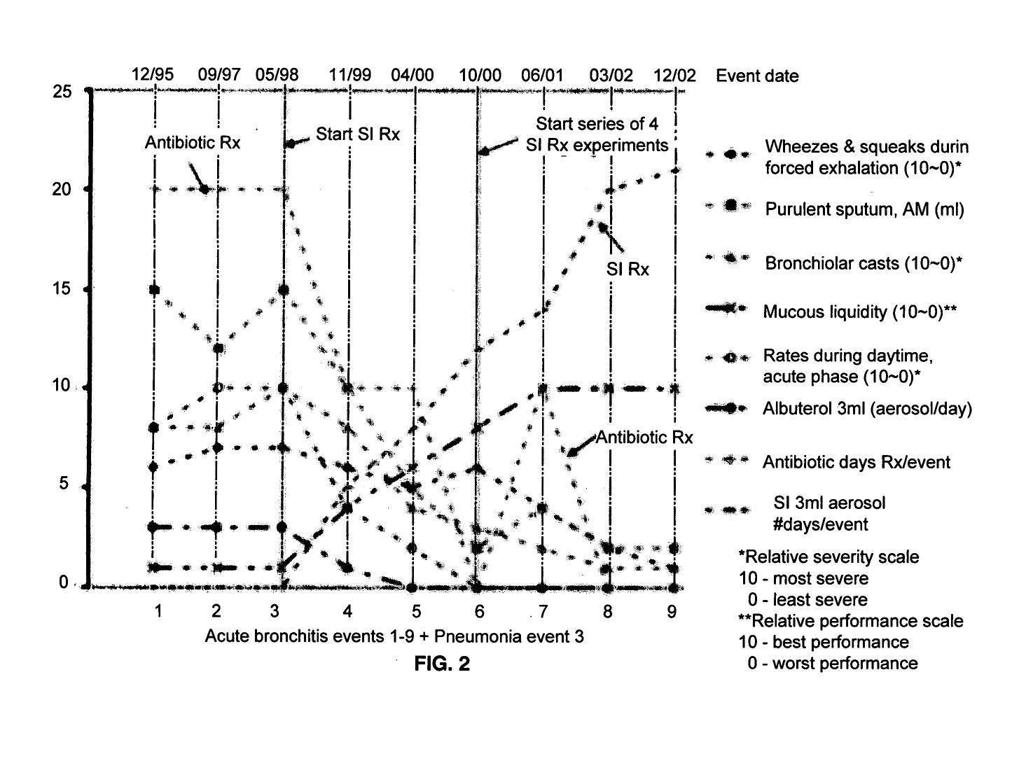

FIG. 2 shows a lung mucosa effects graph presenting what is interpreted to be the benefit of increase in surfactant mucociliary transport efficiency being directly correlated with SI aerosol use across all of the experiments. So called “mucolysis” in CO-ILD treatment is really concerned with mucociliary transport efficacy; what appears to be the missing dynamic is adequate surfactant production essential to facilitate rapid clearance of normal mucous of low viscosity with the normal mucous performing its natural fomite collection function, flowing purposefully and efficiently transported by the cilia without mucous desiccation. Rapid clearance of mucous prevents mucous drying, thickening, and becoming culture medium for microorganism colonies. Note also that SI aerosol use is inversely correlated with a statistically significant decrease in night sweats, purulent sputum production, and days of work lost, as well as required use of albuterol and antibiotics.

Added to the benefits of disease reduction, the combination of reduced need for medications (especially antibiotics), reduced cough and sputum annoyance, and increased respiratory reserve provide significant benefit.

### Beyond the ASC Aerosol Cross-Over Experiment:

In spite of the results of the successful ASC aerosol cross-over experiment, the legacy of the IC's chronic bronchitis persisted. Frank CO-ILD reappearance therefore remained a matter of time. With this knowledge that the IC was not “cured” of historic CO-ILD, it was recognized that new acute exacerbations of acute bronchitis would certainly occur and the old ever-worsening CO-ILD progression would be reestablished. Thus in January 2003 the IC agreed that he would be maintained on daily SI aerosol treatments and assessment of his chronic bronchitis condition would be monitored. Between January 2003 and July 2005, the daily maintenance continued with very few days of treatments being missed. Significantly, during 2003, the IC learned that if the regimen was interrupted, such as by over-confidence that “all is well”, CO-ILD conditions did return.

A mucous plug expectorated during a mild bronchitis episode following a brief lapse early on in the once-daily SI aerosol regimen in August, 2003 helps to confirm this position. This mucous plug which resisted dislodging by coughing, but was easily expectorated about 20 minutes after an SI aerosol inhalation. The mucous plug was embedded in a flood of fluid (surfactant). At the time of this relapse (2003), use of SI had been employed daily for 8 months after more than 3 years in cross over treatment had yielded major remission of all previous bronchitis symptoms with the exception of intermittent rales and occasional small clear mucous plugs. The return of mild acute bronchitis after a careless SI lapse indicates the persistence of mucosa pathology that is prone to regression to more serious bronchitic disease states. Within a week of restarting twice daily SI aerosol treatments, the IC's remission was reestablished suggesting 1) significant nutraceutical effect of ASC applied topically to the respiratory mucosa, and 2) need for antibiotic use becomes much more discretionary.

Of course, any long term regimen requiring daily maintenance is difficult to sustain. Soon, the IC found that if he missed a just a few days, lesser signs, such as small mucous plugs, recurred. On these occasions, prompt return to the SI aerosol regimen immediately re-established remission and prevented recurrence of relapse.

Following the episode in August 2003, the IC remained steady on using twice daily inhalation of the nebulized SI aerosol. During the 2 years that followed, the IC experienced total remission of all chronic bronchitis signs and symptoms end-points, including the rales, which were the longest lasting end-point, and which ceased to occur July 2005. In response to this event, the IC agreed to remain on daily inhalations for an additional full year with unrestrained, normal living activity, and if symptoms or signs did not reappear his CO-ILD remission status would be re-assessed. By June 2006, the IC could jog over 4 miles without any cardio-pulmonary dysfunction. Atenolol 50 mg qd which had been prescribed and used to control chronic hypertension since 2000 was the only medication used since the end of the experiment in 2002 other than the daily ASC inhalations.

In June 2006 the question arose: “has the IC achieved a “clinical cure” of CO-ILD?” To challenge this question, in July, 2006, the IC agreed to completely stop inhalation of the ASC aerosol. From Jul. 1, 2006 to the end of June, 2007, no recurrences of active respiratory disease occurred. Coincidentally, the IC did experience an exacerbation of Meniere's disease during the late summer of 2006, which spontaneously had remission two months later.

In June 2007, the IC was invited to join a group team to run the San Francisco Marathon. The IC felt fit to accept the invitation, entered intensive training, achieving individual runs in training up to 16 miles. Long term functional rehabilitation of his respiratory disease was key to his successful performance.

### Observation of Data—Spirometry, Microbiological, and Chest X-Ray Results:

Spirometry: To quantitatively assess the effects of the SI aerosol experiments, spirometry was performed: 1) immediately prior to implementing the empirical and cross-over SI tests; 2) at the time of completion of the empirical and cross-over experiments and prior to start of daily SI aerosol treatment; and 3) on completion of the rigorous exercise stress experiment. These several spirometry records are:
1) 28 May 1999 prior to initiation of SI treatment; lozenge and upper respiratory safety study completed.
2) 10 Dec. 2002 completion of cross-over experiments; prior to commencing daily SI aerosol initiation;
3) 13 Sep. 2007; post “clinical cure”, peak exercise stress, and reinstitution of daily SI.

Table 2 summarizes the spirometry evidence which discloses significant beneficial respiratory performance results for the IC. The FEV1 liters increased from 3.17 liters to 3.33 liters (5% increase) as a result of SI treatments at the time of acute bronchitis exacerbations, and then further increased to 3.81 liters (20+% increase) as a result of daily SI treatments which established a “clinical cure” of the chronic bronchitis that had gradually become progressively worse over the preceeding 4 decades. An even greater percentage increase in vital capacity occurred as revealed by these spirometry measurements. The FVC increased from 3.73 liters to 4.23 liters (13+% increase) by virtue of acute exacerbation SI treatments, followed by increase to a completely normal 4.75 liters (27+% increase) following daily SI treatments for “clinical cure” of chronic bronchitis (which obviously included prevention of acute bronchitis exacerbations).

TABLE 2
Spirometry evidence discloses beneficial respiratory performance results for the IC
Table 2.

| | Forced Expiration | | Forced Vital | |
|---|---|---|---|---|
| | Volume, 1 second | FEV1, % of | Capacity | FVC, % of |
| Spirometry date | (FEV1), Liters | predicted | (FVC), Liters | predicted |
| 1) (pre) 1999 May 28 | 3.17 | 89.45% | 3.73 | 81.33% |
| 2) (mid) 2002 Dec. 10 | 3.33 | 96.0% | 4.23 | 94.0% |
| % improvement | +5.0% | | +13.4% | |
| 3) (post) 2007 Sep. 13 | 3.81 | 132.8% | 4.75 | 117.8% |
| % improvement | +20.2% | | +27.4% | |

### Microbiology

As to the natural history of progressive chronic bronchitis, the IC produced sputum in ever increasing amounts during the course of the four decades preceding the cross-over experiments. Bacteria such as Serratia marcescens were reported by sputum culture, and fungi such as Chrysosporium also grew in the IC respiratory sputum. These organisms were commonly classified as “normal respiratory flora”. Contrary to convention, these are not normal flora as has been demonstrated in this experiment—wherein mucociliary efficiency with rapid mucous clearance resulted in no “sputum” (inspissated mucous) has been produced, and therefore no flora could be cultured, the continuous condition of the IC since the last episode recorded in 2003.

### X-ray

In 1998, the index case was diagnosed with NHL. Of necessity for management of NHL, many CT scans followed. The correlation between NHL and the progress of the SI experiments are summarized in the following table/graph. The table/graph depicts a second benefit of SI inhalation, namely alteration of the natural progress of NHL.

Current x-rays report clearance of all active pulmonary disease except some emphysema residual in the apexes and some residual scarring in the bases. Obviously, this residual has caused hampering of tolerance for intense exercise, yet the IC's pulmonary and NHL conditions have significantly returned to a high quality of life. Current x-rays are particularly noteworthy since the above charted data show that only two courses of Rituximab occurred in 1999 and 2001. No further Rituximab has been required since February 2001.

At this time, NHL treatment benefits remain under study by means of an ongoing cross-over study of the IC's lymphoma disease. Since 2003 there have been 3 such cross-over events (May 2003, July 2006, July 2009) and in each event, with an average cycle of 2 years with SI followed by 1 year abstinence of SI, the IC's NHL is again going into remission following reinstitution of daily SI inhalation.

### A Second CO-ILD IC:

Given the success of the initial chronic obstructive bronchitis IC experiment, a second experiment was conducted in 2003. The IC was a 56 y/o W/F with Stage III CO-ILD observed by chest imaging to have a new lesion in her left upper lobe. Pulmonology consult declared her emphysema component to be too advanced to tolerate either diagnostic biopsy or chemo-radiation therapy. After one month of three SI doses per day, the CO-ILD staging had improved with pronounced improvement in respiratory function and functional increase in exercise tolerance. Repeat pulmonary and thoracic surgery consultations determined that she not only could now tolerate diagnostic surgery, but could even tolerate a pneumonectomy of her left lung if indicated. Following the SI experiment mobilization, the patient became so elated in her improved pulmonary function, she (against advice) flew to Seattle to spend time with her family. She returned 6 months later, but the lesion had metastasized to her mediastinum and had become inoperable, soon followed by her death due to lung cancer.

What is claimed is:

1. A method for treating a tracheo-bronchial-alveolar tract disease in a subject in need thereof, the method comprising the step of:
administering to a subject in need of such treatment a pharmaceutical composition comprising a therapeutically effective amount of ascorbate or a derivative thereof.

2. The method of claim 1, wherein the tracheo-bronchial-alveolar tract disease is selected from the group consisting of chronic inflammatory/obstructive pulmonary diseases, bronchitis and emphysema.

3. The method of claim 1, wherein said composition contains from about 5 g to about 15 g of ascorbate or a derivative thereof per 100 ml.

4. The method of claim 1, wherein said composition is contained in an aerosol inhalant, nebulized inhalant or a lozenge.

5. A nasal or oral inhalant comprising an aqueous carrier having dissolved therein ascorbate or a derivative thereof.

6. The nasal inhalant of claim 5, wherein said aqueous carrier comprises from about 5 to about 15 wt. % of ascorbate.

7. The nasal inhalant of claim 5, further comprising one or more members of the group consisting of corticosteroids, antihistamines, non-steroidal anti-inflammatory agents, anti-cholinergics, expectorants, antibiotics, histamine H₃ receptor antagonists, leukotriene D₄ antagonists, leukotriene inhibitors, P₂Y agonists, syk kinase analogues and vitamin E.

8. The nasal inhalant of claim 5, wherein said non-steroidal anti-inflammatory agent is selected from the group consisting of acetyl salicylic acid, acetaminophen, indomethacin, diclofenac, piroxicam, tenoxicam, ibuprofen, naproxen, ketoprofen, nabumetone, ketorolac, azapropazone, mefenamic acid, tolfenamic acid, sulindac, diflunisal, tiaprofenic acid, podophyllotoxin derivatives, acemetacin, aceclofenac, droxicam, oxaprozin, floctafenine, phenylbutazone, proglumetacin, flurbiprofen, tolmetin, and fenbufen.

9. The nasal inhalant of claim 7, wherein said antibiotic is selected from the group consisting of tetracycline, chlortetracycline, bacitracin, neomycin, to polymyxin, gramicidin, oxytetracycline, chloramphenicol, flofenicol, gentamycin, erythoromycin, clarithromycin, azithromycin, tulathromycincefurpxo, ceftobitem. ceftiofur, defadroxil, amoxicillin, penicillin, amoxicillin combined with a beta-lactamase inhibitor, sulfonamides, sulfacetamide, sulfamethizole, sulfisoxazole, nitrofurazone, and sodium propionate.

10. An inhalable composition suitable for use in a nebulizer comprising a ascorbate wherein said ascorbate is packaged for simultaneous, sequential, or separate inhalation administration of at least one additional medicament comprising a solution or suspension containing at least one member of the group consisting corticosteroids, antihistamines, non-steroidal anti-inflammatory agents, anti-cholinergics, pharmaceutically acceptable zinc salts, expectorants, antibiotics, histamine H₃ receptor antagonists, leukotriene D₄ antagonists, leukotriene inhibitors, P₂Y agonists, syk kinase analogues, vitamin E and combinations of two or more thereof.

11. A method of treatment of an upper or lower respiratory, viral, inflammatory, or obstructive airway disease comprising administration of an effective amount of a composition of claim 10.

12. The method of claim 11, wherein administration is carried out using a nebulizer.

13. A method for treating non-Hodgkin's lymphoma in a subject in need thereof, the method comprising the step of:
administering to the subject in need of such treatment a pharmaceutical composition comprising a therapeutically effective amount of ascorbate or a derivative thereof.

14. The method of claim 13, wherein said composition contains from about 5 g to about 15 g of ascorbate or a derivative thereof per 100 ml.

15. The method of claim 13, further comprising concurrently administering a compound selected from the group consisting of a aza-anthracenedione, rituximab, ibritumomab and tositumomab.

16. The method of claim 13, wherein said composition is administered to respiratory mucosa or lymphatic tissue of said subject.

17. The method of claim 13, wherein said composition is in the form of an aerosol inhalant or a lozenge.
